(19) United States
(12) Patent Application Publication
(10) Pub. No.: US 2007/0225371 A1
(43) Pub. Date: Sep. 27, 2007

(54) Dermatological composition containing alkyl ureas

(76) Inventor: Jerry Zhang, Grayslake, IL (US)

Correspondence Address: Jerry Zhang, 1061 Chadwick Dr., Grayslake, IL, 60030, US

(21) Appl. No.: 11/389,363

(22) Filed: Mar. 25, 2006

(51) Int. Cl. A61K 31/17

(52) U.S. Cl. 514/588

(57) ABSTRACT

This invention relates to a method for topically treating dermal disorders employing a composition comprising a mixture of urea and a safe and effective amount of an alkyl urea in a therapeutically effective ratio. The urea and alkyl urea are blended with a carrier suitable for topical application to dermal tissues. The carrier is in the form of an aqueous liquid, a gel, a cream, a foam, a liposome, a nonaqueous liquid, a nonaqueous gel, or a nonaqueous cream.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

This invention relates to the field of treating dermal disorders such as, for example, severely dry skin condition, xerosis, ichthyosis, and fungal infection of nail. This invention is directed to a new composition for treating the dermal disorders comprising a mixture of urea and a safe effective amount of an alkyl urea in a therapeutically effective ratio. The urea and alkyl urea are blended with a pharmaceutically acceptable carrier suitable for topical application to dermal tissues.

### BACKGROUND OF THE INVENTION

Urea has been long recognized as a cosmetic ingredient in formulations acting as a humectant and moisturizer. High concentrations of urea, such as greater than 40%, are known to have keratolytic activity as well as mild, antimicrobial effect.

There are a numerous patent disclosures concerning use of urea at high concentrations, generally greater than 35% by weight, for treating severely dry skin condition and fungal infection of nail. For example, U.S. Pat. No. 6,281,239 discloses treatment of onychomycosis using compositions containing 40% urea. U.S. Pat. No. 6,743,417 discloses treatment of onychomycosis with 40% urea and an antioxidant.

Elbaum et al., “Effect of Alkylureas on the Polymerization of Hemoglobin S” in Proc. Nat. Acad. Sci. USA, 71:4718-4722 (1974) report that alkyl ureas promote the dissociation of deoxyhemoglobin S through hydrophobic interaction. The effect is proportional to the length of the alkyl chain substantiating the importance of hydrophobic interaction in the polymerization of hemoglobin S.

U.S. Pat. Publication No. 20050272819 discloses use of alkyl ureas for treating acne. Alkyl ureas are showed to be effective for treating acne without side effects associated with current methods of acne treatment.

However, none of these patents and publications discloses a composition comprising a mixture of urea and an alkyl urea with improved efficacy for treating dermal disorders.

Accordingly, there is a need for a dermatological composition with improved efficacy for treating dermal disorders.

### SUMMARY OF THE INVENTION

Generally speaking, in accordance with the present invention, a method and composition for topically treating dermal disorders includes employing a composition comprising a mixture of urea and alkyl urea in a therapeutically effective ratio, blended in a pharmaceutically acceptable carrier.

Accordingly, it is an object of the invention to provide an improved method for treating dermal disorders.

Another object of the invention is to formulate a dermatological composition comprising a mixture of urea and a safe and effective amount of an alkyl urea in a therapeutically effective ratio, blended in a pharmaceutically acceptable carrier with enhanced physiological (drug) effect.

Still other objects and advantages of the invention will, in part, be obvious and will, in part, be apparent from the following detailed description of the preferred embodiments.

### BRIEF DESCRIPTIONS OF THE DRAWINGS

FIG. 1 shows general structure of urea and a urea derivative.

FIG. 2 shows general structure of a mono-substituted alkyl urea.

### DETAILED DESCRIPTION OF THE PREFERRED EMBODIMENTS

The present invention is directed to a new method and composition for topically treating dermal disorders employing a composition comprising a mixture of urea and a safe and effective amount of an alkyl urea in a therapeutically effective ratio blended in a pharmaceutically acceptable carrier.

Dermal disorders, as used herein, mean skin or nail disorders, including, but not limited to, severely dry skin condition, xerosis, ichthyosis, or fungal infection of nail.

The term ‘safe and effective amount’, as used herein, means an amount of an alkyl urea used in the compositions and methods of the present invention, sufficient enough to significantly and positively modify the condition to be treated but low enough to avoid serious side effects, within the scope of sound medical advice.

It has been unexpectedly found that a composition comprising a mixture of urea and alkyl urea has enhanced efficacy when compared to a composition comprising only urea for treating dermal disorders. It has also been discovered that a composition comprising a mixture of urea and alkyl urea is more effective for treating dermal disorders than a composition comprising only alkyl urea.

Urea is a diamide of carbonic acid. Urea has the general formula of H.sub.2 N—C(O)—NH.sub.2, having a molecular weight of 60.06. Urea molecule is hydrophilic and can form hydrogen bonds.

Urea may be present in an amount of at least 15% by weight, at least 35% by weight, or even as much as 55% by weight, preferably 20 to 50% by weight.

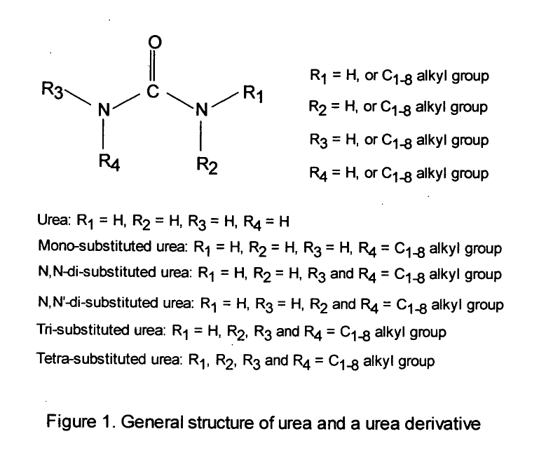

Alkyl ureas are derived from urea by substituting one or more of the hydrogen atoms in the urea molecule with alkyl groups as shown in FIG. 1. The alkyl ureas, as used herein, have the general formula of R.sub.3 R.sub.4 N—C(O)—NR.sub.1 R.sub.2, where R.sub.1, R.sub.2, R.sub.3, and R.sub.4 are each independently hydrogen or C.sub.1-20 alkyl group. The alkyl group can be a straight or branched chain alkyl or a cycloalkyl group.

When one hydrogen atom in urea molecule is substituted by an alkyl group, a mono-substituted alkyl urea is formed. When two hydrogen atoms in urea molecule are substituted by alkyl groups, a di-substituted alkyl urea is formed. There are two types of di-substituted urea: N,N-di-substituted (the two substituted alkyl groups are on the same nitrogen atom) and N,N′-di-substituted (the two substituted alkyl groups are on the different nitrogen atoms) alkyl urea. When three hydrogen atoms in urea molecule are substituted by alkyl groups, a tri-substituted alkyl urea is formed. When all four hydrogen atoms in urea molecule are substituted by alkyl groups, a tetra-substituted alkyl urea is formed.

Examples of the suitable di-substituted alkyl ureas are N,N-dimethyl urea, N,N′-dimethyl urea, N,N-diethyl urea, N,N′-diethyl urea, N,N-bis-(1-butyl)urea, and N,N′-bis-(1-butyl)urea.

Examples of the suitable tri-substituted alkyl ureas are N,N-dimethyl-N′-(1-butyl)urea, N,N-diethyl-N′-(1-butyl)urea, N,N,N′-trimethyl urea, and N,N,N′-triethyl urea.

Examples of the suitable tetra-substituted ureas are N,N,N′,N′-tetramethyl urea, N,N,N′,N′-tetraethyl urea, N,N,N′,N′-tetrakis-(1-butyl)urea.

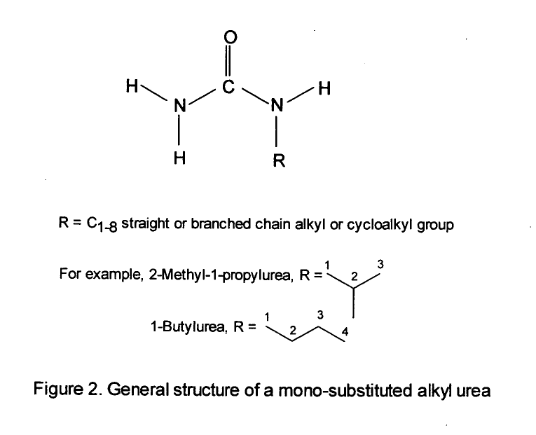

The mono-substituted alkyl ureas have the general formula: R—N(H)—C(O)—NH.sub.2 where R is an alkyl group of 1 to 8 carbon atoms as shown in FIG. 2. The alkyl group can be a straight or branched chain alkyl groups or cycloalkyl group. The straight or branched chain alkyl groups can be saturated (i.e., with no multiple carbon-carbon bonds) or unsaturated (i.e., having double or triple carbon-carbon bonds). Examples of the suitable mono-substituted alkyl ureas are methyl urea, ethyl urea, 1-propyl urea, 2-propyl urea, 1-butyl urea, 2-butyl urea, 2-methyl-1-propyl urea, 1-hexyl urea, 1-octyl urea, cyclohexyl urea, and combinations thereof.

Mono-substituted alkyl ureas with 1 to 8 carbon atoms are preferred alkyl ureas to be used in the present invention.

The safe and effective amount of an alkyl urea present in the urea formulation depends on nature of the dermal (skin or nail) condition to be treated and/or degree of severity of the condition. The alkyl urea may be present in an amount of at least 1% by weight, at least 3% by weight, or even as much as 30% by weight, preferably 5 to 15% by weight.

Urea and alkyl urea can denature proteins by altering their tertiary structure. Urea predominantly denatures proteins through hydrogen bonding while alkyl urea alters protein conformational structure through hydrophobic interactions. Outer layer of human skin (stratum corneum) is hydrophobic, consisting mainly of keratins, a family of fibrous structural proteins. Hydrophobicity of an alkyl urea depends on degree of the alkyl substitution as well as length of the alkyl group. More alkyl substitution and longer alkyl chain increase the hydrophobicity of an alkyl urea. However, more alkyl substitution and longer alkyl chain decrease the moisturizing and hydrogen-bonding activity of an alkyl urea.

It is believed that addition of alkyl urea into the urea composition might provide a unique combination of hydrogen-bonding, keratolytic activity of urea with the lipid disrupting ability of an alkyl urea. It is also believed that a mixture of urea and alkyl urea might provide a unique balance of moisturizing and keratolytic activity. The exact reason for the enhancing effect of the alkyl urea is unknown, but it is believed that the enhancing effect results from a unique combination of moisturizing, hydrogen-bonding, keratolytic and lipid disrupting characteristics of a mixture of urea and alkyl urea.

The ratio of urea to alkyl urea in the composition is important in achieving desired physiological effect by balancing between moisturizing, hydrogen-bonding, and keratolytic activity of urea and hydrophobic interaction of an alkyl urea.

The suitable ratio of urea to alkyl urea in the composition may be from 1:50 to 50:1 by weight, preferably from 1:10 to 10:1 by weight, more preferably from 1:5 to 5:1 by weight.

The composition comprising a mixture of urea and an alkyl urea can be formulated in any pharmaceutically acceptable carrier suitable for topical application to dermal tissues. Aqueous carrier can be water, a gel, a cream, a foam, or a liposome. Nonaqueous-based carrier can be an organic solvent, an organic-based gel, or an organic based cream. A ‘nonaqueous’ carrier is one that is substantially water free. While water is not intentionally added to a nonaqueous carrier, trace amounts of water (for example, existed in the solvent as an impurity) may still be present. It is desired that the amount of water in the nonaqueous carrier be less than about 5% by weight, preferably less than 3% by weight, more preferably less than 1% by weight.

Polyols, also known as polyhydric alcohols, are defined as organic compounds having at least two hydroxyl groups per molecule. The general formula of suitable polyols are: R(OH).sub.n where n is equal to or greater than 2 and R is generally C.sub.2-10 alkyl or substituted alkyl groups.

Polyols are preferred organic solvents as nonaqueous carriers. Examples of the suitable polyols are glycerin (also known as glycerol), propylene glycol (also known as 1,2-propanediol), 1,3-propanediol, 1,2-butanediol, 1,3-butanediol, 1,2-pentanediol, 1,5-pentanediol, 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. Preferred polyols are glycerin, propylene glycol, 1,5-pentanediol, 1,6-hexanediol, and combinations thereof.

Other suitable nonaqueous carriers include polyethylene glycols (PEG) or polypropylene glycols (PPG) of various molecular weights.

The urea/alkyl urea composition of the present invention can contain other ingredients that might provide additional keratolytic/lipid disrupting activity. Examples of the suitable ingredients are alpha- or beta-hydroxy acids. Examples of the suitable alpha-hydroxy acids are glycolic acid, lactic acid, malic acid, citric acid, mandelic acid, or tartaric acid. Examples of the suitable beta-hydroxy acids are salicylic acid, or lactobionic acid. They may be present in an amount of at least 1% by weight, 10% by weight, or even as much as 30% by weight.

The urea/alkyl urea composition of the present invention can contain one or more of conventional antifungal agents. Examples of the suitable antifungal agents are amoroline, clotrimazole, econozole, ketoconazole, miconazole, or ciclopirox. They may be present in an amount of at least 0.1% by weight, 1% by weight, or even as much as 15% by weight.

The urea/alkyl urea composition of the present invention can contain conventional amounts of one or more other desirable ingredients: antioxidants, skin penetration enhancers, surfactants, emulsifiers, or herbal extracts, thickeners, and even sunscreens. Examples of the suitable antioxidants are: vitamin E, vitamin A, lipoic acid, or co-enzyme Q-10. Examples of the suitable vitamin E are tocopherols, tocotrienols, and their derivatives such as, for example, tocopherol acetate, tocopherol palmitate, tocopherol nicotinate, or tocopherol succinate. Examples of the suitable vitamin A are retinol, retinoic acid, structural isomers of retinoic acid (e.g., isotretinoin), esters of retinoic acid such as, for example, retinyl acetate or retinyl palmitate. They may be present in an amount of at least 0.01% by weight, 1% by weight, or even as much as 10% by weight.

In treatment of dermal disorders, the urea/alkyl urea composition is applied at least once a day in an amount to cover the affected area. The duration of the treatment depends on the extent and degree of severity of the dermal conditions.

The following examples are included for purposes of illustrating the technology covered by this disclosure. They are not intended to be exhaustive or to limit the scope of the claimed invention in any manner. One skilled in the art will understand that there are alternatives to these specific embodiments that are not completely described by these examples.

### EXAMPLE 1 (for Comparison)

This example is to prepare a urea composition at 25% concentration.

| Component | Amount (weight percentage) |
|---|---|
| Water | 59.5% |
| Propylene glycol | 15% |
| Urea | 25% |
| Xanthan gum | 0.5% |

The water and propylene glycol were combined at room temperature. Urea was added to the mixture. The mixture was kept stirring until urea was dissolved. Then, xanthan gum, a thickener, was added to the solution in sifted fashion. The mixture was stirred until homogenized, yielding a clear gel.

The urea concentration of 25% by weight is equal to about 4.2 M (molar concentration) based on the molecular weight of urea (M.W.=60.06). At this molar concentration, although it is still an effective moisturizer, the keratolytic or protein denaturing effect of urea is believed to be minimal. It is known that about 6 to 7 M concentration of urea, which is equal to about 36 to 42% (by weight), is minimally needed to show any noticeable keratolytic or protein denaturing activity. Most of over-the-counter (OTC) or prescription urea products for treating dermal disorders have urea concentrations of 40% or greater. Thus, this sample should show minimal keratolytic activity.

### EXAMPLE 2

This example is to demonstrate preparation of a new composition comprising a mixture urea and an alkyl urea in a water-based carrier. The composition in accordance to the present invention was prepared as follows:

| Component | Amount (weight percentage) |
|---|---|
| Water | 55.5% |
| Propylene glycol | 15% |
| Urea | 21% |
| 1-Butyl urea | 8% |
| Xanthan gum | 0.5% |

The water and propylene glycol were combined at room temperature. 1-Butyl urea is available from a number of suppliers such as, for example, Lancaster Synthesis (Ward Hill, Mass.). Urea and 1-butyl urea were added to the mixture. The mixture was kept stirring until urea and 1-butyl urea were dissolved. Then, xanthan gum was added to the solution in sifted fashion. The mixture was stirred until homogenized, yielding a clear gel.

The molecular weight of urea is 60.06 and the molecular weight of 1-butyl urea is 116.16. It can be calculated that the composition from this example contains about 3.5 M of urea and 0.7 M of 1-butyl urea.

In their thermal denaturation studies of protein RNase A in alkyl urea solutions, Poklar et al., “Thermodynamic Stability of Ribonuclease A in Alkylurea Solutions and Preferential Solvation Changes Accompanying its Thermal Denaturation: A Calorimetric and Spectroscopic Study” in Protein Science, 8:832-840 (1999) report that the thermal stability of RNase A in solution decreases with increasing concentrations of denaturants and the size of the hydrophobic group substituted on the urea molecule. It shows that alkyl urea is much more effective in denaturing RNase A in solution than urea.

Based on the temperature profiles of conformational transition (denaturation) of protein RNase A in urea or in 1-butyl urea solution, it can be estimated that 0.7 M of 1-butyl urea is equivalent to about 3.5 M of urea in terms of their protein denaturing capability. These studies provide a general guideline on estimating relative keratolytic activity of urea vs. 1-butyl urea in a dermatological composition.

The total molar concentration of this composition is 4.2 M (3.5 M of urea and 0.7 M of 1-butyl urea), which is same as that in the composition in EXAMPLE 1. However, in terms of efficacy, 0.7 M of 1-butyl urea is estimated to be equivalent to about 3.5 M of urea. Thus, in terms of efficacy for treating dermal disorders, this composition is believed to be acting more like a 7.0 M urea (3.5 M urea added+3.5 M urea equivalent from 1-butyl urea) composition than a 4.2 M urea composition. At this equivalent concentration, which is about 42% of urea by weight, noticeable keratolytic activity could be observed.

### EXAMPLE 3

This example is to compare the relative efficacy of the compositions prepared in EXAMPLES 1 and 2.

Samples from EXAMPLES 1 and 2 were topically applied twice daily to two dry areas of the skin of the lower right leg of one male subject (the inventor herein) in about same amount. The sample from EXAMPLE 1 was applied to one area of the leg. The sample from EXAMPLE 2 was applied to the other area of the leg. After 4 weeks of treatment, the area treated with the sample from EXAMPLE 2 showed noticeable improvement in skin smoothness and appearance, which were clearly visible by comparison between the two treated areas of the leg, whereas the area treated with the sample from EXAMPLE 1 showed little if any improvement.

The observation provides evidence that the composition from EXAMPLE 2 show noticeable keratolytic activity in addition to the moisturizing activity. It shows that the composition comprising a mixture of urea and an alkyl urea is more effective for treating dermal disorders than the composition comprising only urea.

### EXAMPLE 4

This example is to demonstrate preparation of a urea/alkyl urea composition at higher active concentrations (urea at about 5.8 M and 1-butyl urea at about 0.9 M). The composition in accordance to the present invention was prepared as follows:

| Component | Amount (weight percentage) |
|---|---|
| Water | 39.5% |
| Propylene glycol | 15% |
| Urea | 35% |
| 1-Butyl urea | 10% |
| Xanthan gum | 0.5% |

The water and propylene glycol were combined at room temperature. Urea and 1-butyl urea were added to the mixture. The mixture was kept stirring until urea and 1-butyl urea were dissolved. Then, xanthan gum was added to the solution in sifted fashion. The mixture was stirred until homogenized, yielding a clear gel.

### EXAMPLE 5 (For Comparison)

This example is to demonstrate preparation of a composition at 45% urea, which is equal to about 7.5 M.

| Component | Amount (weight percentage) |
|---|---|
| Water | 39.5% |
| Propylene glycol | 15% |
| Urea | 45% |
| Xanthan gum | 0.5% |

The water and propylene glycol were combined at room temperature. Urea was added to the mixture. The mixture was kept stirring until urea was dissolved. Then, xanthan gum was added to the solution in sifted fashion. The mixture was stirred until homogenized, yielding a clear gel.

### EXAMPLE 6

This example is to compare the relative efficacy of the compositions prepared in EXAMPLES 4 and 5.

Samples from EXAMPLES 4 and 5 were topically applied twice daily to two dry areas of the skin of the lower left leg of one male subject (the inventor herein) in about same amount. The sample from EXAMPLE 4 was applied to one area of the leg. The sample from EXAMPLE 5 was applied to the other area of the leg. After 3 weeks of treatment, the area treated with the sample from EXAMPLE 4 showed more significant improvement in skin smoothness and appearance than the area treated with the sample from EXAMPLE 5. The difference in the results was clearly visible by comparison of the two treated areas of the leg.

The observation provides evidence that the composition from EXAMPLE 4 is more effective than the sample prepared from EXAMPLE 5 for treating dry skin conditions.

### EXAMPLE 7

This example is to demonstrate preparation of a urea/alkyl urea composition in a nonaqueous carrier in the form of a solution. The composition in accordance to the present invention was prepared as follows:

| Component | Amount (weight percentage) |
|---|---|
| Propylene glycol | 71% |
| Urea | 21% |
| 1-Butyl urea | 8% |

The propylene glycol was heated to 65.degree. C. Urea and 1-butyl urea were added together to the propylene glycol while maintaining the temperature at 65.degree. C., while stirring until dissolved. The mixture was allowed to cool to room temperature. The mixture was a clear solution.

### EXAMPLE 8

This example is to demonstrate preparation of a urea/alkyl urea composition in a nonaqueous carrier in the form of a cream. The composition in accordance to the present invention was prepared as follows:

| Component | Amount (weight percentage) |
|---|---|
| Propylene glycol | 55% |
| Urea | 35% |
| 1-Butyl urea | 10% |

The propylene glycol was heated to 65.degree. C. Urea and 1-butyl urea were added together to the propylene glycol while maintaining the temperature at 65.degree. C., while stirring until dissolved. The mixture was allowed to cool to room temperature. The mixture was solidified to form a white cream.

It will thus be seen that the objects set forth above, among those made apparent from the preceding description, are efficiently attained and, since certain changes may be made in carrying out the above process and in the composition set forth without departing from the spirit and scope of the invention, it is intended that all matter contained in the above description shall be interpreted as illustrative and not in a limiting sense.

It is also to be understood that the following claims are intended to cover all of the generic and specific features of the invention herein described and all statements of the scope of the invention which, as a matter of language, might be said to fall there between.

Particularly it is to be understood that in the claims, ingredients or compounds recited in the singular are intended to include compatible combinations of such ingredients wherever the sense permits.

What is claimed is:

1. A method for treating dermal disorders employing a composition comprising a mixture of urea and a safe and effective amount of an alkyl urea in a pharmaceutically acceptable carrier, wherein the urea and alkyl urea are in a ratio therapeutically effective for treating the dermal disorders.

2. The method of claim 1 wherein the alkyl urea comprises mono-substituted alkyl urea.

3. The method of claim 2 wherein the alkyl group in the mono-substituted alkyl urea has 1 to 8 carbon atoms.

4. The method of claim 1 wherein the alkyl urea is selected from the group consisting of methyl urea, ethyl urea, 1-propyl urea, 2-propyl urea, 1-butyl urea, 2-butyl urea, and combinations thereof.

5. The method of claim 1 wherein the alkyl urea is 1-butyl urea.

6. The method of claim 1 wherein the urea is in an amount of about 15% to about 55% by weight of the total composition.

7. The method of claim 1 wherein the alkyl urea is in an amount of about 1% to about 30% by weight of the total composition.

8. The method of claim 1 wherein the ratio of urea to alkyl urea is at least about 1 to 50 by weight and no more than about 50 to 1 by weight.

9. A composition for treating dermal disorders comprising a mixture of urea and a safe and effective amount of an alkyl urea in a pharmaceutically acceptable carrier, wherein the urea and alkyl urea are in a ratio therapeutically effective for treating the dermal disorders.

10. The composition of claim 9 wherein the ratio of urea to alkyl urea is at least about 1 to 50 by weight and no more than about 50 to 1 by weight.

11. The composition of claim 9 wherein the urea is in an amount of about 15% to about 55% by weight of the total composition.

12. The composition of claim 9 wherein the alkyl urea is in an amount of about 1% to about 30% by weight of the total composition.

13. The composition of claim 9 wherein the alkyl urea comprises mono-substituted alkyl urea.

14. The composition of claim 13 wherein the alkyl group in the mono-substituted alkyl urea has 1 to 8 carbon atoms.

15. The composition of claim 9 wherein the alkyl urea is selected from the group consisting of methyl urea, ethyl urea, 1-propyl urea, 2-propyl urea, 1-butyl urea, 2-butyl urea, and combinations thereof.

16. The composition of claim 9 wherein the alkyl urea is 1-butyl urea.

17. The composition of claim 9 wherein the pharmaceutically acceptable carrier is a water-based carrier in the form of water, a gel, a cream, a foam, or a liposome.

18. The composition of claim 9 wherein the pharmaceutically acceptable carrier is a nonaqueous-based carrier in the form of an organic solvent, a gel, or a cream.

19. The composition of claim 9 further comprising one or more ingredients selected from the group consisting of vitamin A and derivatives thereof, vitamin E and derivatives thereof, alpha-hydroxy acids, beta-hydroxy acids, antifungal agents, and combinations thereof.

20. A method for treating dermal disorders comprising applying to dermal tissues of a patient in need of such treatment an effective amount of a composition comprising a mixture of urea and a safe and effective amount of an alkyl urea in a pharmaceutically acceptable carrier, wherein the urea and alkyl urea are in a ratio therapeutically effective for treating the dermal disorders, and the urea is in an amount of about 15% to about 55% by weight of the total composition and the alkyl urea is in an amount of about 1 to about 30% by weight of the total composition.
